(19) United States
(12) Patent Application Publication
(10) Pub. No.: US 2008/0154294 A1
(43) Pub. Date: Jun. 26, 2008

(54) Gas Insufflation Device

(76) Inventor: Horst Semm, Gruenwald (DE)

Correspondence Address: ST. ONGE STEWARD JOHNSTON & REENS, LLC, 986 BEDFORD STREET, STAMFORD, CT, 06905-5619, US

(21) Appl. No.: 11/849,866

(22) Filed: Sep. 4, 2007

Related U.S. Application Data
(63) Continuation of application No. PCT/EP2006/002040, filed on Mar. 6, 2006.

(30) Foreign Application Priority Data
Mar. 4, 2005 (DE) 10 2005 010 062.7

(51) Int. Cl. A61B 17/34

(52) U.S. Cl. 606/185

(57) ABSTRACT

The invention relates to a gas insufflation device, comprising a trocar having a tubular trocar sleeve being attached to a trocar body, as well as a trocar pin which may be inserted into the trocar sleeve through the trocar body, characterized in that the inner cross-section of the trocar sleeve is shaped geometrically different with respect to the outer cross-section of the trocar pin in order to form at least one flow channel.

### CROSS-REFERENCE TO RELATED APPLICATIONS

The present application is a continuation of pending International patent application PCT/EP2006/002040 filed on Mar. 6, 2006 which designates the United States and claims priority from German patent application 10 2005 010 062.7 filed on Mar. 4, 2005, the content of which is incorporated herein by reference.

### FIELD OF THE INVENTION

The invention relates to a gas insufflation device according to the preamble portion of the main claim.

### BACKGROUND OF THE INVENTION

In detail, the invention relates to a gas insufflation device comprising a trocar having a tubular trocar sleeve which is fixed to a trocar body, as well as a trocar pin which may be inserted into the trocar sleeve through the trocar body.

A gas insufflation device of the mentioned type is e.g. known from DE 30 00 218 C1. In minimal-invasive surgery, it serves to establish a pneumoperitoneum and to perform intra-abdominal examinations or operations with this facility.

During the minimal-invasive surgery (MIC), a trocar is inserted into the abdomen through a relatively small cut. Through the trocar, an endoscope, further optical instruments or surgical instruments may then be inserted. Therewith, the possibility arises to establish a pneumoperitoneum. Same requires a supply of a gas volume into the abdomen, the gas volume being large enough to create a sufficient operating field and to immediately balance occurring leakage, e.g. when instruments are exchanged. It may also be required to supply correspondingly large gas amounts e.g. in case of laser welding in the abdomen.

From the state of the art, it is known to form an annular gap between the trocar sleeve and the trocar pin, through which the gas supply is performed. This annular gap, however, is often not sufficient to realize large gas flow amounts of up to 45 l/min or more. Besides the mostly small cross-sectional surface of the annular gap, same also forms a flow resistance which is disadvantageous in case of large gas volumes.

JP 09 140721 A describes a gas insufflation device having a trocar comprising a tubular trocar sleeve into which a trocar pin is guided. In order to form suitable flow channels, the trocar sleeve is formed tubular, while the trocar pin is provided with a longitudinal groove forming the flow channel. A similar construction is shown in WO 01/08573 A. To enhance the discharge from the trocar sleeve, same is provided with recesses.

U.S. Pat. No. 6,656,160 B1 also shows circular trocar sleeves in which instruments having different cross-sections are guided in order to enable a sufficient fluid flow.

From WO 96/01132 A, it is known to provide respectively circular cross-sections for the trocar sleeve and the trocar pin, but to regrade same strongly in their diameters in order to provide an annular flow cross-section.

DE 91 09 909 U1 and WO 2004/032770 A respectively show a general structure of trocars without discussing the problem of a flow channel.

It is an object underlying the present invention to provide a gas insufflation device of the aforementioned kind, which enables a reliable supply of large gas volumes while having a simple structure and a simple and cost-effective producibility.

### SUMMARY OF THE INVENTION

According to the invention, the object is solved by the combination of features of the main claim. The sub-claims show further advantageous embodiments of the invention.

According to the invention, it is thus provided that the inner cross-section of the trocar sleeve is formed geometrically different relative to the outer cross-section of the trocar pin in order to form at least one flow channel.

According to the invention, it is thus provided that an additional flow channel is created, which is formed between the outer wall of the trocar pin and the inner wall of the trocar sleeve in that the cross-sectional shape of the inner cross-section of the trocar sleeve differs from that of the outer cross-section of the trocar pin.

According to the invention, it is thus possible to provide the trocar sleeve, which is manufactured as a thin-walled pipe made of a medically suitable steel, with an arbitrary cross-section, e.g. elliptical, lenticular, square, rhombic, in the shape of a triangle or polygon. In this context, it is obvious that the outer contour is correspondingly rounded or provided with a radius in order to prevent the patient from being injured or damaged. Since the trocar pin has a round, circular outer cross-section, there results at least one additional flow channel which is defined by the relation of the cross-section of the trocar sleeve and the cross-section of the trocar pin. Preferably, also a plurality of flow channels may be provided. It is thus possible to dimension the conduit of the at least one flow channel such that a sufficient gas volume may be passed through.

According to the invention, it is particularly advantageous that only an adaptation of the trocar sleeve is required. This is feasible in the most simple manner, since such trocar sleeves are drawn over a die. Therewith, a particularly economic way of manufacturing is provided. It is therefore not required to deviate from the circular cross-section of the trocar pin which is designed in the form of instruments or optics.

According to the invention, it is thus possible to insufflate gas flows or gas volumes of e.g. 1 to 45 l/min without interference or back-pressure.

In a particularly advantageous embodiment of the invention, it is provided that the trocar pin is provided with an insertion part at its distal end portion, the cross-section of said insertion part being formed corresponding to the inner cross-section of the trocar sleeve. If the insertion part or distal end portion of the trocar pin is located in the distal mouth portion of the trocar sleeve, same may consequently be inserted into the abdomen without any problem, without any danger to injure the patient or without any interference of or impact on the surgical operation. The distal end portion of the trocar pin may thus be provided with lugs, the geometry and size of which correspond exactly to the at least one flow channel, and which cover or seal same during the insertion process, in order to prevent an undesired insertion of tissue or the like. After complete insertion of the trocar, the trocar pin is then further advanced, whereby its distal end portion with the insertion part departs from the distal end portion of the trocar sleeve, such that the at least one flow channel is cleared.

The distal end portion of the trocar pin is preferably shaped in the form of a trocar tip (conical or three-edged), wherein the swellings or lugs which serve, in the manner described above, to close the at least one flow channel, connect to the trocar tip smoothly and without steps.

According to the invention, it is possible to shape the trocar sleeve symmetrically or asymmetrically. Consequently, different cross-sectional variants result. Particularly advantageous embodiments are provided if the circular cross-section of the trocar pin is correspondingly guided or centered in the trocar sleeve. Therewith, a seizure is securely prevented.

According to the invention, it may be particularly preferable to shape the trocar sleeve not round, as it was discussed above, i.e. for example triangular, square or in any other shape. Therewith, the twisting reliability is secured, which, in addition, prevents or hinders a slipping or sliding of the trocar sleeve during an operation, such that an additional advantageous effect results.

According to the invention, the distal end of the trocar sleeve may be formed straight or bevelled, such that common basic shapes of trocar sleeves may be used.

In order to guarantee an optimised supply of gas, it may be preferred to provide the trocar body with a gas inlet pipe which substantially opens out directly into the at least one flow channel. In this context, it may be beneficial to form an inlet chamber or the like in order to provide a fluidic advantageous inlet of the gas into the flow channel(s). Also an axial inclination of the gas inlet pipe contributes to this effect.

### BRIEF DESCRIPTION OF THE DRAWINGS

In the following, the invention is described on the basis of embodiments in connection with the drawing, wherein:

FIG. 1 shows a schematic side view of a trocar having a trocar sleeve and a trocar pin in a triangular embodiment,

FIG. 2 shows a front side view of the arrangement according to FIG. 1,

FIG. 3 shows an enlarged partial view of the end portion of a related trocar pin,

FIG. 4 shows a view analogous to FIG. 1 in a square shape,

FIG. 5 shows a front view of the arrangement of FIG. 4,

FIG. 6 shows a front end portion of the related trocar pin with its distal end portion,

FIG. 7 shows a side view of a further embodiment, analogous to FIGS. 1 and 4, in an elliptic shape,

FIG. 8 shows a face side view of the arrangement of FIG. 7,

FIG. 9 shows a view of the related trocar pin with its distal end portion corresponding to the embodiments of FIGS. 7 and 8,

FIG. 10 shows a schematic side view of an embodiment of the trocar pin according to the invention, and

FIG. 11 shows an arrangement, analogous to FIGS. 1, 4 and 7, in section with an assignment of the trocar pin.

### DETAILED DESCRIPTION OF THE INVENTION

According to the invention, a trocar sleeve 1 is provided, which is connected to a trocar body 2, e.g. by means of a screw connect (not shown in detail). This corresponds to the state of the art. A gas inlet pipe 6 is fixed to the trocar body 2, the pipe serving to supply gas.

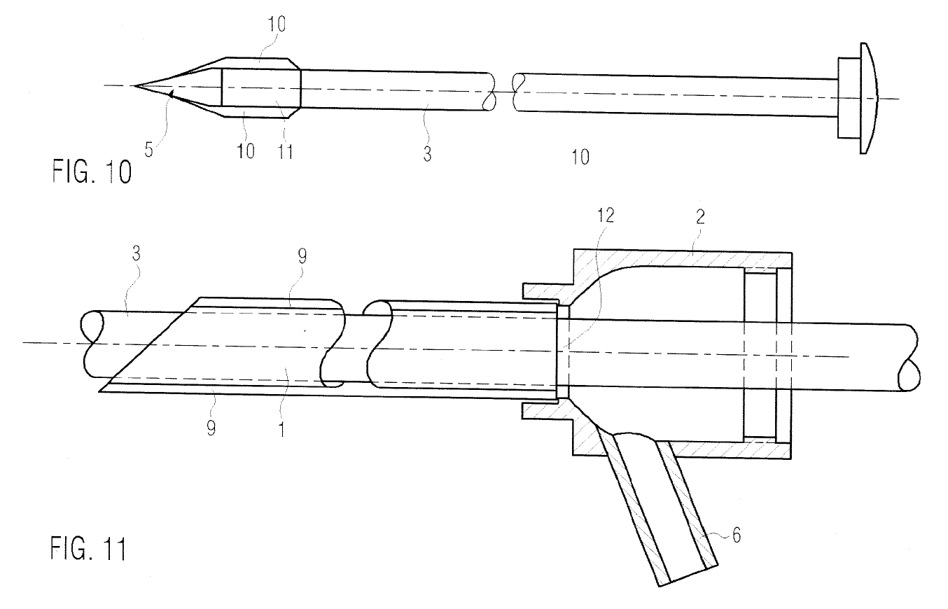

As shown in FIG. 11, a trocar pin (see FIG. 10) may be inserted into the trocar sleeve 1 through the trocar body 2. This also corresponds to the state of the art; a depiction of the required diaphragms or sealing means is omitted for the sake of clarity.

The trocar pin 3 has a round, circular outer cross-section, as is e.g. shown in FIGS. 3, 6, 9 and 10. As discussed, the trocar pin 3 may be formed in different shapes, as is required in minimal-invasive surgery. FIG. 2 for example shows an embodiment having an optic comprising a lens 7 and an optical fiber bundle 8.

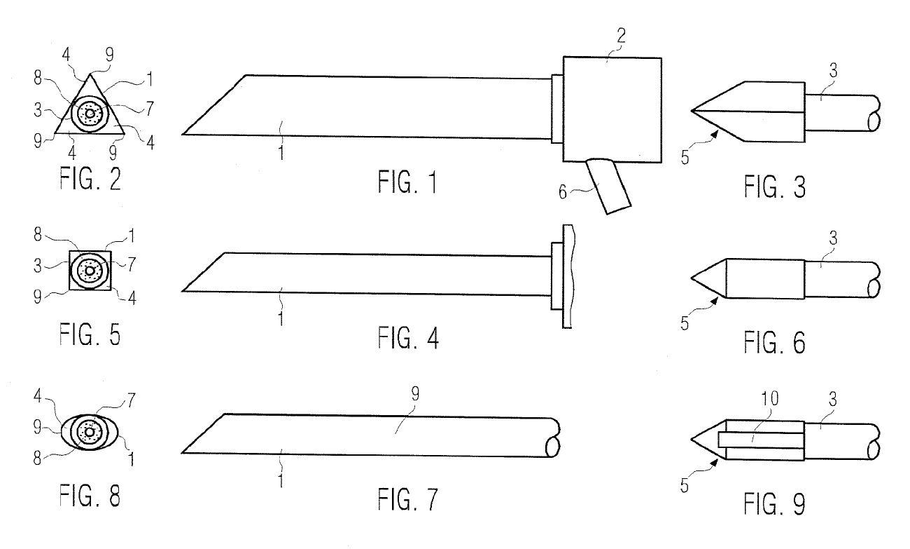

FIGS. 1 to 3 show a first embodiment in which a trocar sleeve 1 is provided, which has a triangular cross-section (equilateral triangle). Consequently, there result three convexities 9 which are rounded and respectively form the corners of the triangle. With these convexities 9, respectively one flow channel is formed, as is shown in FIG. 2. A round trocar pin 3 is therewith guided in the triangular trocar sleeve. FIG. 3 shows a distal end portion 5 of a trocar pin 3, the end portion also having a triangular cross-section and fitting into the triangular cross-section of the trocar sleeve 1 such that its interior is covered or sealed when the trocar sleeve 1 is inserted, such that no tissue may be displaced into the flow channel 4. Upon a further axial feed of the trocar pin 3, the distal end portion 5 leaves the distal end of the trocar sleeve 1, such that the flow channels 4 are cleared.

A further embodiment is shown in FIGS. 4 to 6, in which the trocar sleeve 1 has a square cross-section. Also herein, the circular cross-section of the trocar pin 3 is centered and guided, as is shown in FIG. 5. It is obvious that the related trocar pin 3 (see FIG. 6) is provided with a distal end portion 5 or a tip, which is square and corresponds to the interior of the square cross-section of the trocar sleeve 1.

FIGS. 7 to 9 show a further embodiment. Herein, the cross-section of the trocar sleeve 1 is lenticular such that two opposing convexities 9 or lugs result, by which the two flow channels 4 are formed. Also in this case, the middle region of the lenticular trocar sleeves 1 guides the trocar pin 3 centrally. It is apparent from the depiction of FIG. 9 that the distal end portion 5 is accordingly provided with a lug 10 to seal or cover the two flow channels 4 of the lenticular cross-section upon insertion. FIG. 10 shows an enlarged view of FIG. 9 (rotated by 90°). Herein, the two lugs 10 are again shown in more detail.

FIG. 11 shows an enlarged view in section of the embodiment of FIGS. 7 to 10. Herein, it results in particular that the gas may be supplied through the gas inlet pipe 6 into the interior the trocar body 2 such that same may inflow unresisted into the flow channels 4 (not shown in FIG. 11) which are formed by the convexities 9. For this purpose, an inlet chamber 12 is shown schematically in FIG. 11.

1. A gas insufflation device, comprising a trocar having a tubular trocar sleeve being attached to a trocar body, as well as a trocar pin having a circular cross-section, which may be inserted into the trocar sleeve through the trocar body, characterized in that the inner cross-section of the trocar sleeve is shaped not round and geometrically different with respect to the circular outer cross-section of the trocar pin in order to form at least one flow channel and that the trocar pin is provided with an insertion part at its distal end portion, the cross-section of said insertion part being formed corresponding to the inner cross-section of the trocar sleeve.

2. The device of claim 1, characterized in that the distal end portion is formed in the shape of a trocar tip.

3. The device of claim 1, characterized in that the trocar pin has a circular cross-section.

4. The device of claim 1, characterized in that the trocar sleeve has an asymmetrical cross-section.

5. The device of claim 1, characterized in that the trocar sleeve has a symmetrical cross-section.

6. The device of claim 1, characterized in that the trocar sleeve has a substantially lenticular cross-section.

7. The device of claim 1, characterized in that the trocar sleeve has a substantially square shape.

8. The device of claim 1, characterized in that the trocar sleeve has a substantially rhombic shape.

9. The device of claim 1, characterized in that the trocar sleeve has a substantially triangular shape.

10. The device of claim 1, characterized in that the trocar sleeve has a substantially polygonal shape.

11. The device of claim 1, characterized in that the distal end of the trocar sleeve is formed straight.

12. The device of claim 1, characterized in that the distal end of the trocar sleeve is formed bevelled.

13. The device of claim 2, characterized in that the trocar body is provided with a gas inlet pipe which substantially opens out directly into the at least one flow channel.

14. The device of claim 13, characterized in that the gas inlet pipe opens out into an inlet chamber which opens out into the at least one flow channel.
